(19) United States
(12) Patent Application Publication
(10) Pub. No.: US 2022/0233857 A1
(43) Pub. Date: Jul. 28, 2022

(54) IMPLANTABLE STIMULATOR WITH EXTERNAL DEVICE

(71) Applicant: ResMed Pty Ltd, Bella Vista, NSW (AU)

(72) Inventors: Peter James Dassos, Sydney (AU); Benjamin Peter Johnston, Sydney (AU); Nathan Zersee Liu, Sydney (AU); Sakeena De Souza, Sydney (AU); Mark Neil Phillips, Sydney (AU); Genevieve Claire Madafiglio, Sydney (AU)

(21) Appl. No.: 17/615,019

(22) PCT Filed: May 28, 2020
(86) PCT No.: PCT/IB2020/055091

§ 371 Date: Nov. 29, 2021

Related U.S. Application Data
(60) Provisional application No. 62/855,487, filed on May 31, 2019.

(51) Int. Cl. A61N 1/36; A61N 1/378; A61N 1/372

(52) U.S. Cl. CPC A61N 1/3611; A61N 1/36139; A61N 1/37235; A61N 1/36171; A61N 1/3787; A61N 1/3615

(57) ABSTRACT

A system for aiding a user includes a stimulator, a sensor, a memory, and a control system. The stimulator is configured to be positioned in the user adjacent to an airway of the user. The sensor is configured to generate data associated with the airway of the user. The memory stores machine-readable instructions. The control system includes one or more processors configured to execute the machine-readable instructions to determine, based at least on an analysis of the generated data, that the user is currently experiencing an apnea event. In response to the determination that the user is currently experiencing an apnea event, the control system causes the stimulator to provide electrical stimulation, at a first intensity level, to one or more muscles of the user that are adjacent to the airway to aid in stopping the apnea event.

### CROSS REFERENCE TO RELATED APPLICATIONS

This application claims the benefit of, and priority to, U.S. Provisional Patent Application No. 62/855,487, filed May 31, 2019, which is hereby incorporated by reference herein in its entirety.

### TECHNICAL FIELD

The present disclosure relates to treatment of respiratory-related disorders and more specifically to systems and methods with implantable stimulators and corresponding external devices for addressing one or more types of apnea events.

### BACKGROUND

Various systems exist for aiding users experiencing sleep apnea and related respiratory disorders. Some such systems rely on the user to wear a mask that aids in suppling pressurized air to the airway of the user. Some users find such systems to be uncomfortable, difficult to use, expensive, aesthetically unappealing, etc.

Thus, a need exists for alternative systems and methods for addressing sleep apnea and related respiratory disorders. The present disclosure is directed to solving these problems and addressing other needs.

### SUMMARY

According to some implementations of the present disclosure, a method for aiding a user includes receiving, from one or more sensors, data associated with an airway of the user. The method also includes analyzing the data to determine if the user is experiencing an apnea event, if the user is about to experience and apnea event, if the user is no longer experiencing an apnea event, or any combination thereof. The method further includes in response to a determination that the user is experiencing an apnea event or the user is about to experience an apnea event, causing a stimulator to provide electrical stimulation to a portion of the user to aid in stopping or preventing the apnea event.

According to some implementations of the present disclosure, a system for aiding a user includes a housing, a stimulator, a receiver, a collar, a transmitter, a sensor, a memory, and a control system. The housing is configured to be positioned in the user adjacent to an airway of the user. The stimulator is coupled to the housing. The receiver is coupled to the housing. The collar is configured to be worn around a neck of the user. The transmitter is coupled to the collar and is configured to communicate with the receiver to cause the stimulator to selectively provide electrical stimulation to (i) one or more muscles of the user that are adjacent to the airway (ii) one or more nerves associated with the one or more muscles, or (iii) both (i) and (ii). The sensor is configured to generate data associated with the airway of the user. The memory stores machine-readable instructions. The control system includes one or more processors configured to execute the machine-readable instructions to analyze the generated data. The analysis of the data is for determining (i) if the user is experiencing an apnea event, (ii) if the user is about to experience an apnea event, (iii) if the user is no longer experiencing an apnea event, (iv) or any combination thereof. In response to a determination that (i) the user is experiencing an apnea event or (ii) the user is about to experience an apnea event, the control system causes the transmitter to communicate with the receiver such that the stimulator provides the electrical stimulation to aid in stopping or preventing the apnea event.

According to some implementations of the present disclosure, a method includes receiving, from one or more sensors, data associated with an airway of the user. The method also includes determining that the user is currently experienced an apnea event based at least in part on the received data. The method further includes in response to determining that the user is currently experiencing an apnea event, causing a stimulator to provide electrical stimulation, at a first intensity level, to one or more muscles of the user that are adjacent to the airway to aid in stopping the apnea event.

According to some implementations of the present disclosure, a system for aiding a user includes a stimulator, a sensor, a memory, and a control system. The stimulator is configured to be positioned in the user adjacent to an airway of the user. The sensor is configured to generate data associated with the airway of the user. The memory stores machine-readable instructions. The control system includes one or more processors configured to execute the machine-readable instructions to determine, based at least on an analysis of the generated data, that the user is currently experiencing an apnea event. In response to the determination that the user is currently experiencing an apnea event, the control system causes the stimulator to provide electrical stimulation, at a first intensity level, to one or more muscles of the user that are adjacent to the airway to aid in stopping the apnea event.

According to some implementations of the present disclosure, a method includes receiving, from one or more sensors, data associated with the user. The method also includes analyzing the data to determine if the user is currently experiencing a first type of apnea event and analyzing the data to determine if the user is currently experiencing a second type of apnea event that is different from the first type of apnea event. The method further includes responsive to determining that the user is currently experiencing the first type of apnea event, causing a first stimulator to provide electrical stimulation to one or more muscles of the user that are adjacent to a throat of the user to aid in stopping the first type of apnea event. The method additionally includes responsive to determining that the user is currently experiencing the second type of apnea event, causing a second stimulator to provide electrical stimulation to a diaphragm of the user to aid in stopping the second type of apnea event.

According to some implementations of the present disclosure, a system for aiding a user in breathing during sleep includes a first stimulator, a second stimulator, one or more sensors, a memory, and a control system. The first stimulator is configured to be positioned in the user adjacent to a throat of the user. The second stimulator is configured to be positioned in the user adjacent to a diaphragm of the user. The one or more sensors is configured to generate data. The memory stores machine-readable instructions. The control system includes one or more processors configured to execute the machine-readable instructions to analyze the generated data to determine if the user is currently experiencing a first type of apnea event. The control system further analyzes the generated data to determine if the user is currently experiencing a second type of apnea event that is different than the first type of apnea event. In response to a determination that the user is currently experiencing the first type of apnea event, the control system causes the first stimulator to provide electrical stimulation to one or more muscles of the user that are adjacent to the throat of the user to aid in stopping the first type of apnea event. In response to a determination that the user is currently experiencing the second type of apnea event, the control system causes the second stimulator to provide electrical stimulation to the diaphragm of the user to aid in stopping the second type of apnea event.

The foregoing and additional aspects and implementations of the present disclosure will be apparent to those of ordinary skill in the art in view of the detailed description of various embodiments and/or implementations, which is made with reference to the drawings, a brief description of which is provided next.

### BRIEF DESCRIPTION OF THE DRAWINGS

The foregoing and other advantages of the present disclosure will become apparent upon reading the following detailed description and upon reference to the drawings.

FIG. 1A is a diagram that illustrates an overview of a respiratory system of a user;

FIG. 1B is a diagram that illustrates an upper airway of the user of FIG. 1A;

FIG. 2 is a block diagram of a system for aiding a user (e.g., in breathing during sleep), according to some implementations of the present disclosure;

FIG. 3A is a perspective view of a system with a stimulator (positioned in the user) and an external device (unrolled) in the form of a collar for aiding a user (e.g., in breathing during sleep), according to some implementations of the present disclosure;

FIG. 3B is a perspective view of the system of FIG. 3A where the external device is worn/donned by the user;

FIG. 4A is a perspective view of a user wearing a system with two stimulators (positioned in the user) and two external devices, one external device in the form of a collar and the other external device in the form of a band/belt, for aiding the user (e.g., in breathing during sleep), according to some implementations of the present disclosure; and

FIG. 4B illustrates the system of FIG. 4A relative to a cross-sectional diagram view of the user to better illustrate the positioning of the two stimulators in the user, according to some implementations of the present disclosure.

While the present disclosure is susceptible to various modifications and alternative forms, specific embodiments have been shown by way of example in the drawings and will be described in detail herein. It should be understood, however, that the present disclosure is not intended to be limited to the particular forms disclosed. Rather, the present disclosure is to cover all modifications, equivalents, and alternatives falling within the spirit and scope of the present disclosure as defined by the appended claims.

### DETAILED DESCRIPTION

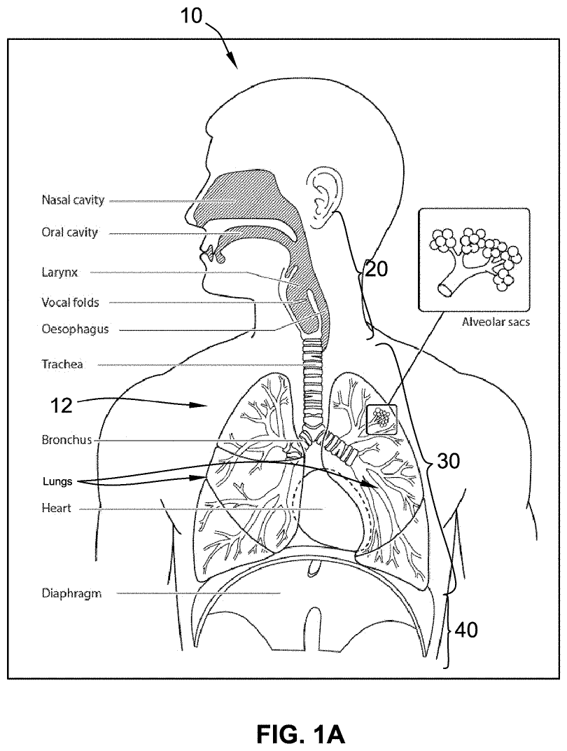

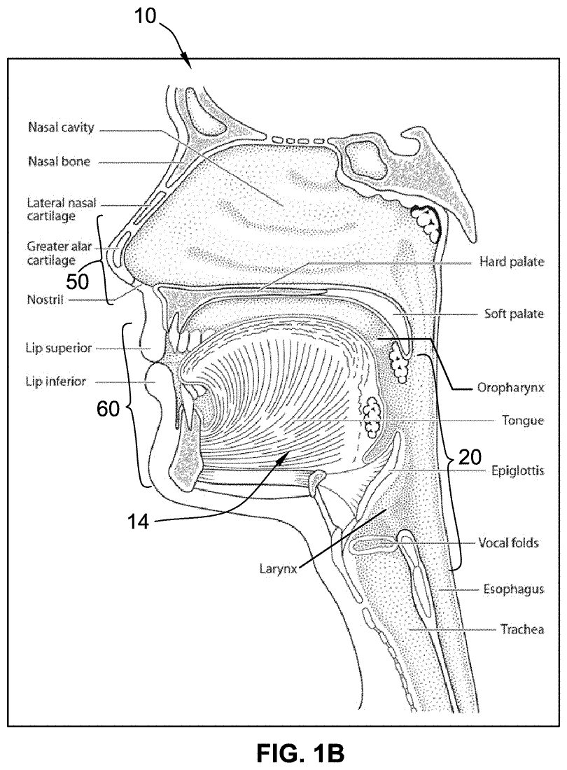

Referring to FIG. 1A, an overview of a respiratory system 12 of a user 10 (e.g., patient) is shown, which generally includes a nasal cavity, an oral cavity, a larynx, vocal folds, an oesophagus, a trachea, a bronchus, lungs, alveolar sacs, a heart, and a diaphragm. More generally, the user 10 has a throat 20, which includes a region(s) of the respiratory system 12 of the user 10 generally in the neck area of the user 10. The diaphragm of the user 10 is a sheet of muscle that extends across the bottom of the rib cage of the user 10. The diaphragm generally separates the thoracic cavity 30 of the user 10, which contains the heart, lungs, and ribs, from the abdominal cavity 40 of the user 10. As the diaphragm contracts, the volume of the thoracic cavity 30 increases and air is drawn into the lungs.

As is described below in greater detail, one or more stimulators of the present disclosure can be placed (e.g., implanted via surgery, injected via syringe, etc.) inside the user 10 to aid the user 10, for example, in breathing while sleeping. For example, one or more stimulators can be placed in the throat 20 of the user 10 (e.g., adjacent to one or more nerves innervating the muscles of the neck/throat and/or the diaphragm, and/or contacting one or more muscles in the neck/throat 20 of the user 10), in the thoracic cavity 30 and/or the abdominal cavity 40 (e.g., adjacent to and/or contacting the diaphragm of the user 10), or a combination thereof.

Referring to FIG. 1B, a view of an upper airway 14 of the user 10 is shown, which includes the nasal cavity, nasal bone, lateral nasal cartilage, greater alar cartilage, nostrils (one shown), a lip superior, a lip inferior, the larynx, a hard palate, a soft palate, an oropharynx, a tongue, an epiglottis, the vocal folds, the oesophagus, and the trachea.

The respiratory system 12 of the user 10 facilitates gas exchange. The nose 50 and mouth 60 of the user 10 form the entrance to the airways of the user 10. As best shown in FIG. 1A, the airways include a series of branching tubes, which become narrower, shorter, and more numerous as they penetrate deeper into the lungs of the user 10. The prime function of the lungs is gas exchange, allowing oxygen to move from the inhaled air into the venous blood and carbon dioxide to move in the opposite direction. The trachea divides into right and left main bronchi, which further divide eventually into terminal bronchioles. The bronchi make up the conducting airways, and do not take part in gas exchange. Further divisions of the airways lead to the respiratory bronchioles, and eventually to the alveoli. The alveolated region of the lungs is where the gas exchange takes place, and is referred to as the respiratory zone.

A range of respiratory disorders exist that can impact the user 10. Certain disorders are characterized by particular events (e.g., apneas, hypopneas, hyperpneas, or any combination thereof). Examples of respiratory disorders include Obstructive Sleep Apnea (OSA), Cheyne-Stokes Respiration (CSR), respiratory insufficiency, Obesity Hyperventilation Syndrome (OHS), Chronic Obstructive Pulmonary Disease (COPD), Neuromuscular Disease (NMD), and Chest wall disorders.

Obstructive Sleep Apnea (OSA) is a form of Sleep Disordered Breathing (SDB) and is characterized by events including occlusion and/or obstruction of the upper air passage during sleep. OSA results from a combination of an abnormally small upper airway and the normal loss of muscle tone in the region of the tongue, soft palate, and posterior oropharyngeal wall during sleep. The condition causes the affected patient to stop breathing for periods typically of 30 to 120 seconds in duration, sometimes 200 to 300 times per night. OSA often causes excessive daytime somnolence, and it may cause cardiovascular disease and brain damage. The syndrome is a common disorder, particularly in middle aged overweight males, although a person affected may have no awareness of the problem.

Cheyne-Stokes Respiration (CSR) is another form of sleep disordered breathing. CSR is a disorder of a user's respiratory controller in which there are rhythmic alternating periods of waxing and waning ventilation known as CSR cycles. CSR is characterized by repetitive de-oxygenation and re-oxygenation of the arterial blood. It is possible that CSR is harmful because of the repetitive hypoxia. In some users, CSR is associated with repetitive arousal from sleep, which causes severe sleep disruption, increased sympathetic activity, and increased afterload.

Respiratory failure is an umbrella term for respiratory disorders in which the lungs are unable to inspire sufficient oxygen or exhale sufficient CO₂ to meet the user's needs. Respiratory failure may encompass some or all of the following disorders.

A user with respiratory insufficiency (a form of respiratory failure) may experience abnormal shortness of breath on exercise.

Obesity Hyperventilation Syndrome (OHS) is the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common. These include increased resistance to air movement, extended expiratory phase of respiration, and loss of the normal elasticity of the lung. Examples of COPD are emphysema and chronic bronchitis. COPD is caused by chronic tobacco smoking (primary risk factor), occupational exposures, air pollution and genetic factors. Symptoms include: dyspnea on exertion, chronic cough and sputum production.

Neuromuscular Disease (NMD) is a broad term that encompasses many diseases and ailments that impair the functioning of the muscles either directly via intrinsic muscle pathology, or indirectly via nerve pathology. Some users suffering from NMD are characterized by progressive muscular impairment leading to loss of ambulation, being wheelchair-bound, swallowing difficulties, respiratory muscle weakness and, eventually, death from respiratory failure. Neuromuscular disorders can be divided into rapidly progressive and slowly progressive: (i) rapidly progressive disorders: characterized by muscle impairment that worsens over months and results in death within a few years (e.g. amyotrophic lateral sclerosis (ALS) and duchenne muscular dystrophy (DMD) in teenagers); (ii) variable or slowly progressive disorders: characterized by muscle impairment that worsens over years and only mildly reduces life expectancy (e.g. limb girdle, Facioscapulohumeral and myotonic muscular dystrophy). Symptoms of respiratory failure in NMD include: increasing generalized weakness, dysphagia, dyspnea on exertion and at rest, fatigue, sleepiness, morning headache, and difficulties with concentration and mood changes.

Chest wall disorders are a group of thoracic deformities that result in inefficient coupling between the respiratory muscles and the thoracic cage. The disorders are usually characterized by a restrictive defect and share the potential of long term hypercapnic respiratory failure. Scoliosis and/or kyphoscoliosis may cause severe respiratory failure. Symptoms of respiratory failure include: dyspnea on exertion, peripheral oedema, orthopnea, repeated chest infections, morning headaches, fatigue, poor sleep quality and loss of appetite.

According to some implementations of the present disclosure, a system (e.g., system 100, 200, 300) is provided to aid users (e.g., patients) experiencing respiratory events (e.g., apnea events) during sleep. An apnea typically occurs when air flow for a user falls below a predetermined threshold for a duration (e.g. 10 seconds). A first type of apnea event is called an obstructive apnea. Obstructive apneas typically occur when, despite user effort to breathe, some obstruction of the airway does not allow air to flow. A second type of apnea event is called a central apnea. Central apneas typically occur when an apnea is detected that is due to a reduction in breathing effort, or the absence of breathing effort, despite the airway being patent (e.g., open). A third type of apnea event is called a mixed apnea. Mixed apneas typically occur when a reduction or absence of breathing effort coincides with an obstructed airway.

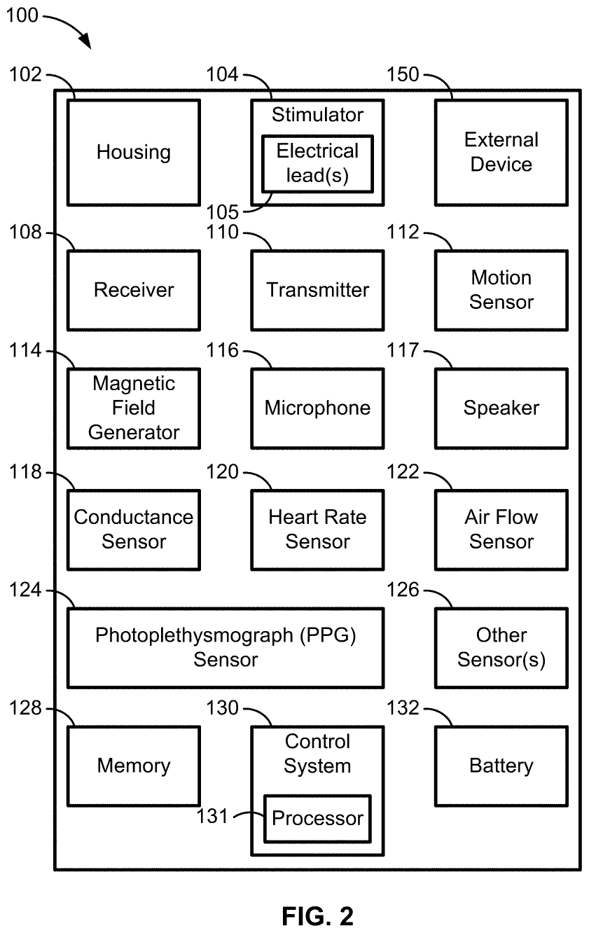

Referring to FIG. 2, a block diagram of a system 100 for aiding a user (e.g., user 10 in FIGS. 1A and 1B) is shown. The system 100 can aid the user 10 (i) in breathing while sleeping, (ii) in breathing while awake, (iii) in opening an airway of the user, (iv) in starting or increasing a breathing function (e.g., contracting a diaphragm), (v) or any combination thereof. In some implementations, the system 100 aids the user 10 by causing one or more muscles of the user 10 to contract to (i) open an airway of the user 10, (ii) to cause the user 10 to inhale air (e.g., breathing effort), or (iii) both.

The system 100 includes one or more of: a housing 102, a stimulator 104, a receiver 108, a transmitter 110, a motion sensor 112, a magnetic field generator 114, a microphone 116, a conductance sensor 118, a heart rate sensor 120, an air flow sensor 122, a photoplethysmography (PPG) sensor 124, one or more other sensors 126 (e.g., EKG sensor, EEG sensor, EMG sensor, blood flow sensor, respiration sensor, pulse sensor, etc.), a memory 128, a control system 130, a battery 132, an external device 150, or any combination(s) thereof. That is the system 100 can include any portion of and any combination of these elements and the elements can be combined in various different arrangements (e.g., physical and/or wireless) and/or housings.

Some of the elements of the system 100 are positioned in the user 10 (e.g., implanted in the user 10) and others of the elements of the system 100 are positioned outside the user 10 (e.g., worn/donned by the user 10). One or more of the elements of the system 100 that are positioned in the user 10 can be so positioned by being injected into the user 10 using, for example, a syringe with a hypodermic needle attached thereto. Alternatively or additionally, one or more of the elements of the system 100 that are positioned in the user 10 can be so positioned by being surgically placed therein (e.g., cutting open the skin and positioning the element(s) therein and suturing the skin closed).

The stimulator 104 is positioned in the user 10 such that one or more electrical leads 105 of the stimulator 104 are positioned adjacent to one or more muscles of the user 10 and/or one or more nerves of the user 10 that are connected to the one or more muscles of the user 10. In some implementations, the one or more electrical leads 105 includes a first electrical lead 105 that is positioned to stimulate a first one of the one or more muscles and/or a first one of the one or more nerves. Similarly, a second electrical lead 105 is positioned to stimulate a second one of the one or more muscles and/or a second one of the one or more nerves. In some implementations, the first electrical lead 105 provides the electrical stimulation at a first frequency and the second electrical lead 105 provides the electrical stimulation at a second frequency that is different from the first frequency. In some implementations, the first electrical lead 105 provides the electrical stimulation at a first intensity and the second electrical lead 105 provides the electrical stimulation at a second intensity that is different from the first intensity. Alternatively, the stimulator 104 may be leadless, with the stimulator body being conductive and the ends of the body acting as electrodes.

Once the stimulator 104 is positioned in the user 10, the stimulator 104 is capable of delivering electrical and/or magnetic stimulation to the user 10 to aid in causing the one or more muscles of the user 10 to contract. The contraction of the one or more muscles of the user 10 can aid in opening an airway of the user 10. The contraction can alternatively or additionally aid in causing the user 10 to have breathing effort (e.g., causing the diaphragm to draw/suck in air).

The electrical stimulation can be applied directly to the one or more muscles of the user 10 (e.g., muscles in the throat 20 of the user 10, muscles surrounding and/or adjacent to an airway of the user 10, the diaphragm of the user 10, etc. or any combination thereof) and/or directly to the one or more nerves that are connected to the one or more muscles. Directing the electrical stimulation to the one or more nerves (as opposed to the one or more muscles directly) allows for a relatively lower intensity (e.g., voltage, amperage, etc. or any combination thereof) of the electrical stimulation to be applied to cause the one or more muscles (connected to the one or more nerves) to contract.

The stimulator 104 includes or is an electrical conductor (e.g., one or more electrically conductive wires with or without a portion being electrically insulated). The stimulator 104 includes the one or more electrical leads 105, which are capable of carrying and/or flowing and delivering electrical current to the one or more muscles and/or one or more nerves of the user 10. The electrical current can be supplied by the battery 132 or other power source that is directly and physically connected to the stimulator 104. The battery 132 can be rechargeable. In some implementations, the battery 132 can be recharged by the magnetic field generator 114 and/or the external device 150. Alternatively to the stimulator 104 including the battery 132, in some implementations, the electrical current is supplied wirelessly by the magnetic field generator 114 (which can be included in the external device 150) directly to the electrical conductor(s).

In some implementations, the stimulator 104 only includes one or more electrically conductive wires, with or without a portion being electrically insulated. In some such implementations, the stimulator 104/wire has a length between about 1 millimeter and about 100 centimeters; between about 1 millimeter and about 100 millimeters; between about 1 millimeter and about 10 millimeters; or any length therebetween. Further, in some such implementations, the stimulator 104/wire has a diameter between about 0.01 millimeters and about 5 millimeters; between about 0.1 millimeter and about 2 millimeters; between about 0.1 millimeter and about 1 millimeter; or any diameter therebetween. The size and shape of the stimulator 104 can be selected to permit the injection of the stimulator 104 into the user 10 via a syringe with an attached hypodermic needle.

In some implementations, the stimulator 104 is directly positioned in the user 10. In such implementations, the housing 102 is not required. Alternatively, the stimulator 104 or a portion thereof is coupled to the housing 102 (e.g., positioned at least partially therein) and the housing 102 (with the stimulator 104 coupled thereto) is positioned in the user 10. The housing 102 can have the shape of an elongated pill (or any other shape) that is conducive to being injected into the user 10 using, for example, a syringe with a hypodermic needle attached thereto. In some implementations, the housing 102 electrically insulates at least a portion of the stimulator 104 (e.g., the entire stimulator 104 except for the one or more electrical leads 105 or conductive ends) from surrounding tissue of the user 10.

In addition to the stimulator 104 being coupled to the housing 102, a number of other elements of the system 100 can be coupled to the housing 102 and placed into the user 10. For example, in some implementations, the receiver 108, the motion sensor 112, the microphone 116, the conductance sensor 118, the heart rate sensor 120, the air flow sensor 122, the photoplethysmography (PPG) sensor 124, the other sensor(s) 126, the memory 128, the control system 130, the battery 132, or any combination thereof can be coupled to the housing 102 and positioned in the user 10 along with the stimulator 104. By coupled to the housing 102 it is meant that the element coupled to the housing 102 is completely incased within the housing 102, attached to an exterior surface of the housing 102, partially protruding from one or more openings in the housing 102, directly or indirectly attached to the housing 102, or any combination thereof.

For example, the stimulator 104 and the receiver 108 are coupled to the housing 102 and positioned in the user 10. For another example, the stimulator 104, the receiver 108, and the PPG sensor 124 are coupled to the housing 102 and positioned in the user 10. For yet another example, the stimulator 104, the receiver 108, the PPG sensor 124, the memory 128, and the control system 130 are coupled to the housing 102 and positioned in the user 10. Various other combinations of elements being coupled to the housing 102 and positioned in the user 10 are contemplated.

In some implementations, the receiver 108 is coupled to the housing 102 and/or the stimulator 104. The receiver 108 is able to receive communications (e.g., signals) from the transmitter 110. The transmitter 110 can be coupled to and/or positioned within the external device 150. The communications received by the receiver 108 can cause the stimulator 104 to provide the electrical stimulation to the one or more muscles of the user 10 and/or the one or more nerves of the user 10. In some implementations, the receiver 108 and the transmitter 110 enable wireless communication between the stimulator 104 and the external device 150. In some implementations, the communications are indicative of instructions to cause the stimulator 104 to deliver electrical stimulation. In some implementations, the receiver 108 and/or the transmitter 110 are referred to as wireless control elements (e.g., wireless control elements 235).

Various sensors can be included in the system 100 for generating data that can be analyzed by the control system 130 and/or by one or more other systems (e.g., mobile phones, computers, servers, cloud based devices, etc.) to determine information and/or to make decisions regarding the application and/or cessation of electrical stimulation to be applied to the user 10 via the stimulator 104.

In some such implementations, the system 100 includes the motion sensor 112. The motion sensor 112 can include one or more accelerometers, one or more gyroscopes, or any combination thereof. The motion sensor 112 can be used to generate motion data that is indicative of breathing or a lack thereof by the user 10.

In some implementations, the motion sensor 112 can be coupled to the housing 102 and positioned in the user 10. Alternatively, the motion sensor 112 can be separate from the housing 102 and/or the stimulator 104 and positioned in the user 10. In such implementations where the motion sensor 112 is positioned in the user 10, the motion sensor 112 can be positioned adjacent to the airway of the user 10 to generate data associated with movements or lack of movements of the airway that indicate breathing or a lack thereof. The positioning of the motion sensor 112 can be in the throat 20 (FIGS. 1A and 1B) of the user 10, the thoracic cavity 30, the abdominal cavity 40, or a combination thereof.

In some other implementations, the motion sensor 112 can be coupled to the external device 150 (e.g., a collar, a band/belt, etc., or any combination thereof) and worn by the user 10. In the implementations where the external device 150 is a collar that is configured to be worn/donned about a neck and/or throat 20 of the user 10, the motion sensor 112 can be coupled to the external device 150 such that the motion sensor 112 is positioned adjacent to a portion of the airway of the user 10 when the external device 150 is worn/donned around the neck/throat 20 of the user 10. As such, the motion sensor 112 is positioned to generate motion data indicative of breathing or a lack thereof by the user 10 (e.g., moving, expanding, retracting, etc. of the neck/throat 20 adjacent to an airway indicates breathing).

Similarly, in the implementations where the external device 150 is a band and/or belt that is configured to be worn/donned about a chest and/or abdomen of the user 10, the motion sensor 112 can be coupled to the external device 150 such that the motion sensor 112 is positioned adjacent to the chest and/or abdomen of the user 10 when the external device 150 is worn/donned by the user 10. As such, the motion sensor 112 is positioned to generate motion data indicative of breathing or a lack thereof by the user 10 (e.g., moving, expanding, retracting, etc. of the chest and/or abdomen indicates breathing).

In addition to, or in lieu of, the motion sensor 112, the system 100 can include the microphone 116, the conductance sensor 118, the heart rate sensor 120, the air flow sensor 122, the PPG sensor 124, the other sensor(s) 126, or any combination thereof, where such sensors or portion thereof is in the user 10 and/or coupled to the external device 150 in the same, or similar, fashion as described above for the motion sensor 112.

For example, in some implementations, the system 100 includes the PPG sensor 124 coupled to the external device 150 such that the PPG sensor 124 is positioned adjacent to the throat 20, or on the neck of the user 10, when the external device 150 is worn/donned by the user 10 as a collar. In such implementations, the PPG sensor 124 is able to generate data that is indicative of blood flow of the user adjacent to the airway, blood oxygen levels of the user adjacent to the airway, heart rate of the user, an apnea event the user is currently experiencing, an apnea event the user is likely to experience in the future, or any combination thereof.

For another example, in some implementations, the system 100 includes the microphone 116 coupled to the external device 150 such that the microphone 116 is positioned adjacent to the throat 20 and/or neck of the user 10 when the external device 150 is worn by the user 10 as a collar. In such implementations, the microphone 116 is able to generate data (e.g., sound data) that is indicative of snoring, choking, an apnea event the user is currently experiencing, an apnea event the user is likely to experience in the future, or any combination thereof.

For another example, in some implementations, the system 100 includes the speaker 117 coupled to the external device 150. In such implementations, the microphone 116 and the speaker 117 can be combined into an acoustic sensor, as described in, for example, WO 2018/050913, which is hereby incorporated by reference herein in its entirety. In such implementations, the speaker 117 generates or emits sound waves at a predetermined interval and the microphone 116 detects the reflections of the emitted sound waves from the speaker 117. The sound waves generated or emitted by the speaker 117 have a frequency that is not audible to the human ear (e.g., below 20 Hz or above around 18 kHz) so as not to disturb the sleep of the user 10 or a bed partner. Based at least in part on the data from the microphone 116 and/or the speaker 117, the control system 130 can determine movement of the user 10 and/or determine whether the user is or is going to experience an apnea, as described herein.

For another example, in some implementations, the system 100 includes the heart rate sensor 120 coupled to the external device 150 such that the heart rate sensor 120 is positioned adjacent to the chest of the user 10 when the external device 150 is worn by the user 10 as a band/belt. In such implementations, the heart rate sensor 120 is able to generate data that is indicative a heart rate and/or pulse of the user 10.

For another example, in some implementations, the system 100 includes the air flow sensor 122 coupled to the housing 102 such that the air flow sensor 122 is positioned adjacent to and/or at least partially within the airway of the user 10 when the housing 102 is positioned in the user 10. In such implementations, the air flow sensor 122 is able to generate data that is indicative of air flow in the airway of the user 10.

The other sensor(s) 126 that can be included in the system 100 and positioned in the user 10 and/or be coupled to the external device 150 include, for example, a blood oxygen sensor, a blood flow sensor, a pulse sensor, a respiration sensor, an EKG sensor, an EMG sensor, an EEG sensor, a strain gauge, an accelerometer, a capacitive sensor, a strain gauge sensor, an analyte sensor, a moisture sensor, a camera, an infrared (IR) sensor, an ultrasonic oxygen sensor, an electrical oxygen sensor, a chemical oxygen sensor, an optical oxygen sensor, a sphygmomanometer sensor, an oximetry sensor, a galvanic skin response (GSR) sensor or any combination thereof. Each of such other sensor(s) 126 can generate data that can be analyzed by the control system 130 and/or by one or more other systems to determine information and/or to make decisions regarding the application and/or cessation of electrical stimulation to be applied to the user 10 via the stimulator 104.

The memory 128 can include one or more physically separate memory devices, such that one or more memory devices can be coupled to the housing 102 and/or the external device 150. The memory 128 acts as a non-transitory computer readable storage medium on which is stored machine-readable instructions that can be executed by the control system 130 and/or one or more other systems. The memory 128 is also able to store (temporarily and/or permanently) the data generated by the sensors of the system 100. In some implementations, the memory 128 includes non-volatile memory, battery powered static RAM, volatile RAM, EEPROM memory, NAND flash memory, or any combination thereof. In some implementations, the memory 128 is a removable form of memory 128 (e.g., a memory card).

Like the memory 128, the control system 130 can be coupled to the housing 102 and/or the external device 150. The control system 130 is coupled to the memory 128 such that the control system 130 is configured to execute the machine-readable instructions stored in the memory 128. The control system 130 includes one or more processors 131 and/or one or more controllers. In some implementations, the one or more processors 131 includes one or more x86 INTEL processors, one or more processors based on ARM® Cortex®-M processor from ARM Holdings such as an STM32 series microcontroller from ST MICROELECTRONIC, or any combination thereof. In some implementations, the one or more processors 131 include a 32-bit RISC CPU, such as an STR9 series microcontroller from ST MICROELECTRONICS or a 16-bit RISC CPU such as a processor from the MSP430 family of microcontrollers, manufactured by TEXAS INSTRUMENTS.

In some implementations, the control system 130 is a dedicated electronic circuit. In some implementations, the control system 130 is an application-specific integrated circuit. In some implementations, the control system 130 includes discrete electronic components.

The control system 130 is able to receive input(s) (e.g., signals, generated data, instructions, etc.) from any of the other elements of the system 100 (e.g., the sensors, etc.). The control system 130 is able to provide output signal(s) (e.g., via the transmitter 110, via the magnetic field generator 114, via the external device 150, etc.) to cause one or more actions to occur in the system 100 (e.g., to cause the stimulator 104 to provide electrical stimulation to the user 10, etc.).

The control system 130 is able to analyze the data generated by any of the sensors of the system 100 to determine (i) if the user 10 is experiencing an apnea event, (ii) if the user is about to experience an apnea event, (iii) if the user is no longer experiencing an apnea event, (iv) a current sleep state of the user 10, (v) a tension of the one or more muscles of the user 10, (vi) or any combination thereof.

Based on one or more of such determinations, the control system 130 is able to cause the stimulator 104 to provide electrical and/or magnetic stimulation to the user 10 to (i) aid in stopping an apnea event currently being experienced by the user 10 and/or (ii) aid in preventing an apnea event about to be experienced by the user 10. In some such implementations, the control system 130 causes the transmitter 110 to transmit a signal to the receiver 108 to cause the electrical stimulation of the one or more muscles of the user 10. In some other implementations, the control system 130 causes the external device 150 and/or the magnetic field generator 114 to wireless power the stimulator 104 to provide the electrical stimulation to the one or more muscles and/or the one or more nerves of the user 10.

In addition to causing the stimulator 104 to provide the electrical and/or magnetic stimulation, the control system 130 is able to vary one or more parameters of the electrical stimulation provided by the stimulator 104. The one or more parameters of the stimulation include frequency, intensity, duration, dwell time, rise time in a pulse, a ratio of on-time to an off-time, or any combination thereof.

In some implementations, the one or more parameters of the stimulation are varied based on a measured response (e.g., using one or more of the sensors of the system 100) of the one or more muscles to the stimulation. In some implementations, the modification to the parameters can be based on a continuous feedback loop by the control system 130 continuing to analyze the data generated by one or more of the sensors of the system 100 (e.g., the motion sensor 112, the air flow sensor 122, the PPG sensor 124, etc. or any combination thereof). As such, the control system 130 is able to modify (e.g., in real-time, while the user is experiencing the same apnea event, after the user experiences an apnea event but before the user experiences another apnea event, etc.) one or more of the parameters based on the continued analysis.

For example, if the continued analysis of the generated data from one or more of the sensors of the system 100 indicates that the user 10 is still experiencing an apena event in the presence of the stimulation, the control system 130 can cause the stimulator 104 to increase the intensity of the stimulation applied to the user 10. For another example, if the continued analysis indicates that the user 10 is no longer experiencing an apena event after stimulation, the control system 130 can cause the stimulator 104 to stop providing the simulation to the user 10. As such, the user 10 is less likely to be desensitized over time to the stimulation as compared to systems that continually apply stimulation (even when the user is not experiencing an apnea event).

In some implementations, the control system 130 causes the stimulator 104 to automatically increase an intensity of the stimulation applied to the user 10. As such, the intensity is likely to reach a level that causes the one or more muscles of the user 10 to contract without the intensity having to be set artificially high from the beginning of the stimulation.

As discussed above, the control system 130 can continually monitor the generated data to determine if a current level of the automatically increased intensity of the stimulation has caused the one or more muscles of the user 10 to contract. When the control system 130 determines that the current level has not caused the one or more muscles of the user 10 to contract, the control system 130 causes and/or permits the stimulator 104 to continue automatically increasing the intensity of the stimulation beyond the current level. Similarly, when the control system 130 determines that the current level has caused the one or more muscles of the user 10 to contract, the control system 130 causes the stimulator 104 to stop automatically increasing the intensity of the stimulation at the current level. As such, a proper intensity (e.g., not an artificially high intensity, which can be painful) for the stimulation for the user 10 is reached.

As described above, the external device 150 can be in the form of a collar, a band, a belt, etc., or any combination thereof and worn/donned by the user 10. In some such implementations, the external device 150 includes a housing made entirely or at least partially from a stretchable material such that the external device 150 can be at least partially held close to the skin of the user 10 when worn. For example, the collar (e.g., external device 150) can include stretchable material so the collar is snug around the neck of the user 10 (without choking the user 10). As such, when a PPG sensor 124 is included in the collar (e.g., external device 150), the PPG sensor 124 can be held in close relationship to the neck of the user 10, which can aid in providing more accurate data from the PPG sensor 124.

Additionally or alternatively to the collar, band, and belt form factors for the external device 150, the external device 150 can also include and/or be in the form of a patch that is able to be stuck to the skin of the user 10. The external device 150 can also include and/or be a headgear that is configured to be worn about a head of the user 10. Other form factors for the external device 150 are contemplated. For example, the external device 150 include and/or be in the form of a scarf, a shirt, pants, a mobile phone, a tablet, a computer, or any combination thereof.

The external device 150 can include any number of the elements of the system 100. For example, in some implementations, the transmitter 110, the motion sensor 112, the magnetic field generator 114, the microphone 116, the conductance sensor 118, the heart rate sensor 120, the photoplethysmography (PPG) sensor 124, the other sensor(s) 126, the memory 128, the control system 130, the battery 132, or any combination thereof is coupled to and/or positioned within the external device 150. In some implementations, the external device 150 at least includes one or more of the sensors of the system 100, the magnetic field generator 114, the memory 128, the control system 130, and the battery 132. In some implementations, the external device 150 at least includes the transmitter 110, the memory 128, the control system 130, and the battery 132. In some implementations, the external device 150 is plugged directly into a power source (e.g., a wall outlet) such that there is no need for the battery 132 in the external device 150.

As discussed above, the control system 130 is able to determine if a user is experiencing or about to experience one or more types of apneas and to take one or more actions in response thereto. Additionally, the control system 130 is able to determine if a user is experiencing or about to experience one or more other respiratory events and/or respiration related diseases and to take one or more actions in response thereof. Such other respiratory events and/or respiration related diseases as discussed herein include, for example, hypopneas, hyperpneas, sleep disordered breathing, cheyne-stokes respiration, respiratory failure, obesity hyperventilation syndrome, chronic obstructive pulmonary disease, neuromuscular disease, chest wall disorders, or any combination thereof.

The control system 130 executes a respiration event determination algorithm for the determination of the presence of respiration events (e.g., apneas, hypopneas, hyperpneas, etc.). In some implementations, the respiration event determination algorithm receives as an input at least a portion of the data generated from one or more of the sensors of the system 100 and provides as an output a flag that indicates that a respiration event (e.g., an apnea, a hypopnea etc.) has been detected. In some implementations, the respiration event determination algorithm receives as an input at least a portion of the data generated from one or more of the sensors of the system 100 and provides as an output an instruction to activate the stimulator 104 to provide electrical stimulation to the user 10. In some such implementations, the instruction includes instructions for setting at least a portion of the one or more parameters of the electrical stimulation to be provided by the stimulator 104.

In some implementations of the system 100, a respiration event of an apnea is detected when a function of respiratory flow rate (e.g., determined at least partially using the air flow sensor 122) falls below a flow rate threshold for a predetermined period of time. The function may determine a peak flow rate, a relatively short-term mean flow rate, or a flow rate intermediate of relatively short-term mean and peak flow rate, for example an RMS flow rate. The flow rate threshold may be a relatively long-term measure of flow rate.

In some implementations of the system 100, a respiration event of a hypopnea is detected when a function of respiratory flow rate (e.g., determined at least partially using the air flow sensor 122) falls below a second flow rate threshold for a predetermined period of time. The function may determine a peak flow rate, a relatively short-term mean flow rate, or a flow rate intermediate of relatively short-term mean and peak flow rate, for example an RMS flow rate. The second flow rate threshold is greater than the flow rate threshold used to detect apneas.

In some implementations of the system 100, a respiration event of an apnea is detected when a function of blood flow rate (e.g., determined at least partially using the PPG sensor 124) falls below a flow rate threshold for a predetermined period of time. The function may determine a peak flow rate, a relatively short-term mean flow rate, or a flow rate intermediate of relatively short-term mean and peak flow rate. The flow rate threshold may be a relatively long-term measure of flow rate.

The control system 130 executes a snore event determination algorithm for the determination of the presence of snoring related events (e.g., snoring, choking, etc.). In some implementations, the snoring event determination algorithm receives as an input at least a portion of the data generated from one or more of the sensors (e.g., the microphone 116, the motion sensor 112, etc.) of the system 100 and provides as an output (i) a flag that indicates that a snoring event (e.g., an apnea, a hypopnea etc.) has been detected, (ii) a metric of the extent to which snoring is present, or (iii) both (i) and (ii).

In some implementations of the system 100, the snore event determination algorithm may determine an intensity of a flow rate signal in the range of 30-300 Hz. Further, the snore event determination algorithm may filter the respiratory flow rate signal to reduce background noise.

The control system 130 executes an airway patency algorithm for the determination of the patency (e.g., openness) of a user's airway. In some implementations, the airway patency algorithm receives as an input a respiratory flow rate signal (e.g., determined at least partially using the air flow sensor 122) and determines the power of the signal in the frequency range of about 0.75 Hz and about 3 Hz. The presence of a peak in this frequency range is indicative of an open airway. The absence of a peak in this frequency range is indicative of a closed airway. In some implementations, the airway patency algorithm receives as an input a respiratory flow rate signal and determines the presence or absence of a cardiogenic signal. The absence of a cardiogenic signal is indicative of a closed airway.

The control system 130 executes a therapy parameter algorithm for the determination of one or more of the parameters (e.g., intensity, frequency, duration, etc.) of the stimulator 104. In some such implementations, the therapy parameter algorithm receives as an input the output(s) of one or more other algorithms described herein and outputs one or more values for the one or more parameters (e.g., intensity, frequency, duration, etc.) of the electrical stimulation provided by the stimulator 104.

While the system 100 is shown as including one stimulator 104, one external device 150, and one battery 132, it is contemplated that the system 100 can include any number of stimulators 104 (e.g., one, two, three, five, ten, fifty, etc.), the system 100 can include any number of external devices (e.g., one, two, three, five, ten, fifty, etc.), and the system 100 can include any number of batteries 132 (e.g., one, two, three, five, ten, etc.). The ratio of stimulators to external devices can be one-to-one or a different ratio. For example, in some implementations, two or more stimulators 104 can be controlled by and/or communicate with one external device 150.

A method of using the system 100 to aid the user 10 when experiencing an apnea event is now described. The control system 130 (in the external device 150 or in the housing 102) executes a respiration event determination algorithm for the determination of the presence of respiration events in the user 10. In some such implementations, the respiration event determination algorithm is stored as instructions in the memory 128.

The control system 130 analyzes data generated by one or more of the sensors (e.g., the motion sensor 112, the PPG sensor 124, etc.) of the system 100 included in the external device 150 to determine if the user 10 is currently experiencing an apnea event (e.g., an obstructive apnea event). If the control system 130 determines that the user 10 is currently experiencing an apnea event, the control system 130 causes the stimulator 104 to provide stimulation. The stimulation can be provided to one or more muscles and/or one or more nerves of the user 10 that are adjacent to the throat 20 of the user 10. The stimulation can aid in stopping the apnea event (e.g., by causing the one or more muscles in the throat 20 to contract and open the airway of the user 10).

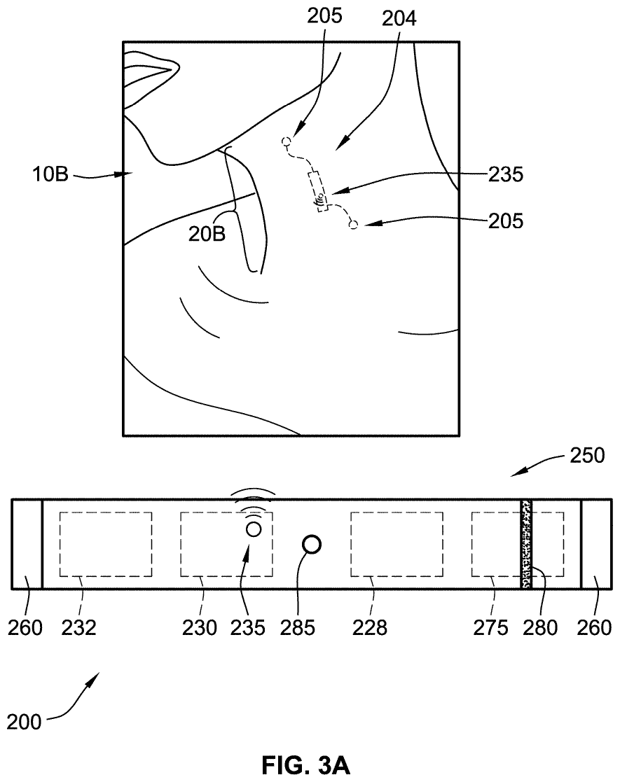

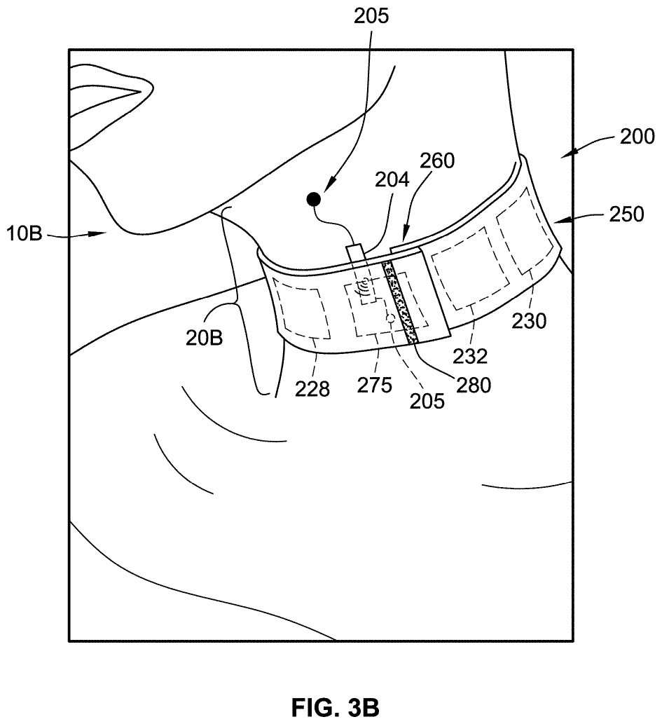

Referring to FIGS. 3A and 3B, a system 200 is shown relative to a user 10B, where an external device 250 (in the form of a collar) is worn by the user 10B in FIG. 3B and removed from the user 10B in FIG. 3A for better illustration of the components of the external device 250. The system 200 is the same as, or similar to, the system 100. The system 200 includes (i) a stimulator 204 positioned in a throat 20B of the user 10B and (ii) the external device 250.

The stimulator 204 is the same as, or similar to, the stimulator 104 described herein in connection with FIG. 2. The stimulator 204 is shown with two electrical leads 205 adjacent to one or more muscles of the user 10B, although any number of electrical leads 205 are contemplated (e.g., one electrical lead, three electrical leads, five electrical leads, ten electrical leads, etc.). Likewise, the stimulator may be leadless with the ends of the stimulator capsule acting as electrodes to deliver stimulation.

The stimulator 204 is shown without a housing for ease of illustration, but just like stimulator 104, the stimulator 204 can be coupled to a housing and/or any other elements described herein (e.g., a receiver, a sensor, a battery, a wireless control element 235). The stimulator 204 is positioned generally in the throat 20B of the user 10B such that the stimulator 204 is positioned to provide electrical stimulation to one or more muscles in the throat 20B and/or the neck of the user 10B. As such, the stimulator 204 can aid in opening an airway of the user 10B.

The external device 250 is in the form of a collar that is wearable by the user 10B around the throat 20A/neck of the user 10B. The external device 250 can be made of any type of material(s) (e.g., one or more types of plastic, one or more types of metal, nylon, one or more types of fabric, stretchable fabric, etc., or any combination thereof) suitable for being worn on a human body (e.g., neck).

The external device 250 can include any type of coupling mechanism 260 (FIG. 3A) to aid in attaching the external device 250 about the neck and/or throat 20B of the user 10B. For example, the coupling mechanism 260 can include a hook and loop fastener, a magnetic clasp, a snap connection, a ball clasp, a bead clasp, a barrel clasp, a fishhook clasp, a push button clasp, a springing clasp, a lobster claw clasp, a hook and loop clasp, etc. or any combination thereof.

In some implementations, the coupling mechanism 260 includes a loop at one end of the external device 250 into which the opposite end of the external device 250 fits through and doubles back to secure to an outside surface of the external device 250 using, for example, hook and loop fasteners. Various other ways of securing the external device 250 about the user 10B are contemplated. In some implementations, the coupling mechanism 260 aids in securing the external device 250 to the user 10B in a snug fashion. Alternatively, the coupling mechanism 260 aids in securing the external device 250 to the user 10B in a loose fashion.

As best shown in FIG. 3A, the external device 250 includes a sensor 275, a memory 228, a control system 230, and a battery 232. The sensor 275 is the same as, or similar to, the motion sensor 112, the microphone 116, the conductance sensor 118, the heart rate sensor 120, the PPG sensor 124, the other sensor(s) 126, or any combination thereof. The memory 228 is the same as, or similar to, the memory 128 described herein in connection with FIG. 2. The control system 230 is the same as, or similar to, the control system 130 described herein in connection with FIG. 2. The battery 232 is the same as, or similar to, the battery 132 described herein in connection with FIG. 2.

The external device 250 (and/or the stimulator 204) can also include one or more wireless control elements 235 such that the external device 250 and the stimulator 204 can wirelessly communicate with and/or wirelessly power the stimulator 204. The one or more wireless control elements 235 can be imbedded/included in the control system 230 and/or be separate therefrom. For example, the external system 250 can include a transmitter that is the same as, or similar to, the transmitter 110 (and the stimulator 204 can include a receiver that is the same as, or similar to, the receiver 108), the magnetic field generator 114, a wireless control module, or any combination thereof. In some implementations, the stimulator 204 itself (e.g., the electrically conductive wire forming at least a portion of the stimulator 204) serves as a wireless receiver without needing any other components. In some implementations, the wireless control element 235 of the stimulator 204 is or includes a receiver (e.g., receiver 108) and the wireless control element 235 of the external device 250 is or includes a transmitter (e.g., transmitter 110).

As shown in FIG. 3B, when the user 10B wears the external device 250 around the throat 20B/neck of the user 10B, the external device 250, or a portion thereof, is directly adjacent to the stimulator 204. As such, in some implementations, wireless communication and/or wireless charging/powering between the external device 250 and the stimulator 204 is enabled. Additionally, such positioning of the external device 250 positions the sensor 275 adjacent to the airway of the user 10B.

In some implementations, indicia can be included on the external device 250 to aid the user 10B in aligning the external device 250 with one or more portions of the anatomy of the user 10B. As such, the sensor 275 can be appropriately placed relative to the user 10B. For example, a vertical line indicium 280 can be included (e.g., printed) on an external surface of the external device 250. The vertical line indicium 280 can indicate to the user 10B a location of the sensor 275 (which can be imbedded and/or otherwise hidden in the external device 250) to be aligned with the user's anatomy (e.g., midline of the throat 20B).

For another example, the external device 250 can include other features to aid the user 10B in aligning the external device 250 when donning the external device 250. For example, a cutout 285 (e.g., having a circular shape, a square shape, a triangular shape, a polygonal shape, etc. or any combination thereof) in the external device 250 can indicate a location of the external device 250 that should be aligned with a specific part of the user's anatomy (e.g., midline of the throat 20B) such that, for example, the sensor 275 is appropriately placed relative to the user 10B.

By appropriately placed, it is meant that the sensor 275 is positioned in a location relative to the user 10B such that the sensor 275 is able to generate reliable and/or usable data. In some such implementations, the location of the sensor 275 depends on the type of sensor(s) included in the sensor 275. For example, if the sensor 275 is a motion sensor, the appropriate location for the sensor 275 maybe be in a first location and if the sensor 275 is a PPG sensor, the appropriate location for the sensor 275 maybe be in a second location that is the same or different from the first location.

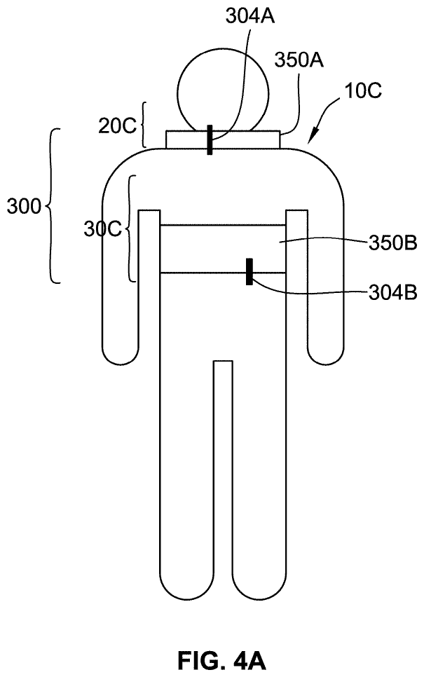

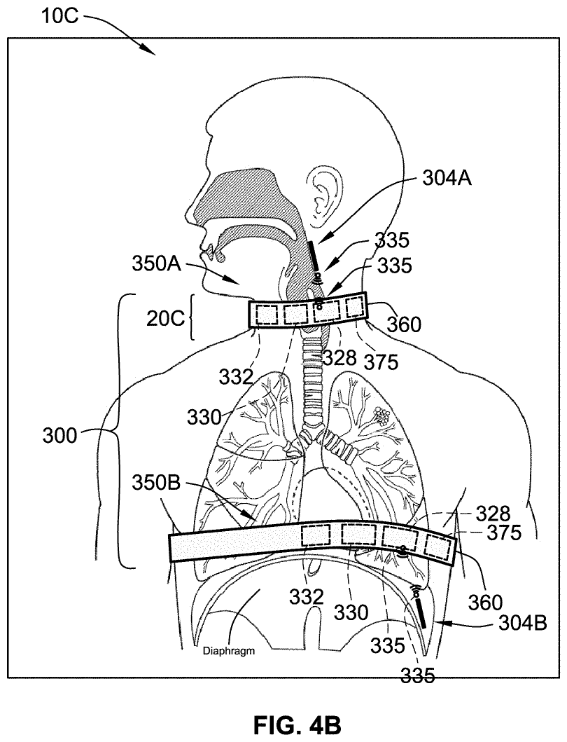

Referring to FIG. 4A, a system 300 is shown relative to a user 10C. The system 300 is the same as, or similar to, the systems 100, 200. The system 300 includes a first external device 350A worn about a throat 20C of the user 10C and a second external device 350B worn about a chest 30C of the user 10C. The first external device 350A can be referred to as a collar and the second external device can be referred to as a chest band. The system 300 also includes a first stimulator 304A positioned in the throat 20C or neck of the user 10C and a second stimulator 304B positioned in an abdominal cavity or a thoracic cavity of the user 10C.

The stimulators 304A and 304B are both the same as, or similar to, the stimulators 104, 204 described herein in connection with FIGS. 2, 3A, and 3B. The external devices 350A and 350B are the same as, or similar to, the external devices 150, 250 described herein in connection with FIGS. 2, 3A, and 3B. The system 300 mainly differs in that the system 300 includes two stimulators and two external devices that work together to aid the user 10C.

Referring to FIG. 4B, the system 300 is shown relative to a cross-sectional diagram view of the user 10C to better illustrate the positioning of the stimulators 304A, 304B in the user 10C. Also shown are more details on the external devices 350a, 350B. As noted above, the first and second external devices 350A, 350B are the same as, or similar to, the external devices 150, 250. Specifically, each of the external devices 350A, 350B includes a sensor 375, a memory 328, a control system 330, a battery 332, a coupling mechanism 360, and a wireless control element 335, which are the same as, or similar to, the sensor 275, the memory 228, the control system 230, the battery 232, the coupling mechanism 260, and the wireless control element 235 of the system 200 described in connection with FIGS. 3A and 3B. The second external device 350B mainly differs in its size relative to the first external device 350A and the external device 250. Namely, the second external device 350B is larger to be wearable about a chest of the user 10C.

In some implementations, the first external device 350A and the first stimulator 304A operate independently from the second external device 350B and the second stimulator 304B. In such implementations, the first external device 350A and the first stimulator 304A form a first sub-system of the system 300 that aid the user 10C in addressing a first type of apnea events (e.g., obstructive apneas) by, for example, causing muscles in the throat 20C to contract to open an airway. Similarly, in such implementations, the second external device 350B and the second stimulator 304B form a second sub-system of the system 300 that aid the user 10C in addressing a second type of apnea events (e.g., central apneas) by, for example, causing the diaphragm of the user 10C to contract to aid breathing effort of the user 10C. In such implementations, both the first external device 350A (collar) and the second external device 350B (chest band) include respective memories 328 and respective control systems 330.

In some other implementations, the first and second external devices 350A, 350B operate together and are coupled together (e.g., wirelessly and/or wired). In some such implementations, only one of the first and second external devices 350A, 350B includes the memory 328 and the control system 330. That is, for example, the second external device 350B (chest band) includes the memory 328, the control system 330, the sensor 375, and the battery 332, and the first external device 350A (collar) includes the sensor 375 and the battery 332. For another example, the first external device 350A (collar) includes the memory 328, the control system 330, the sensor 375, and the battery 332, and the second external device 350B (chest band) includes the sensor 375 and the battery 332.

It should be understood that the sensor 375 in the first external device 350A and the sensor 375 in the second external device 350B can be the same type of sensor(s) or different sensor(s). For example, in some implementations, the sensor 375 in the first external device 350A (collar) is a PPG sensor (e.g., like the PPG sensor 124) and the sensor 375 in the second external device 350B (chest band) is a motion sensor (e.g., like the motion sensor 112).

While the first and second stimulators 304A, 304B are shown as being two separate and distinct stimulators, it is contemplated that the first and second stimulators 304A, 304B can be physically and/or electrically linked. For example, a common housing (not shown) can be implanted in the user 10C (e.g., in the thoracic cavity of the user 10C). From the common housing, one or more electrical leads of the first stimulator 304A can extend into the neck of the user 10C to be adjacent to one or more muscles and/or one or more nerves in the neck of the user 10C. Further, from the common housing, one or more electrical leads of the second stimulator 304B can extend into the abdominal cavity and/or thoracic cavity of the user 10C to be adjacent to the diaphragm of the user 10C. In some such implementations, one or more batteries can be coupled to the common housing for suppling electrical current to the first and second stimulators 304A, 304B.

A method of using the system 300 to aid the user 10C when experiencing one or more types of apnea events is now described. The control system 330 (in the first external device 350A, in the second external device 350B, or a combination thereof) executes a respiration event determination algorithm for the determination of the presence of respiration events in the user 10C. In some such implementations, the respiration event determination algorithm is stored as instructions in the memory 328.

The control system 330 analyzes data generated by the sensor 375 in the first external device 350A to determine if the user 10C is currently experiencing a first type of apnea event (e.g., an obstructive apnea event). The control system 330 also analyzes data generated by the sensor 375 in the second external device 350B to determine if the user 10C is currently experiencing a second type of apnea event (e.g., a central apnea event).

If the control system 330 determines that the user 10C is currently experiencing the first type of apnea event, the control system 330 causes the first stimulator 304A to provide electrical stimulation. The electrical stimulation can be provided to one or more muscles and/or one or more nerves of the user 10C that are adjacent to the throat 20C of the user 10C. The electrical stimulation can aid in stopping the first type of apnea event (e.g., by causing the one or more muscles in the throat 20C to contract and open the airway of the user 10C).

If the control system 330 determines that the user 10C is currently experiencing the second type of apnea event, the control system 330 causes the second stimulator 304B to provide electrical stimulation. The electrical stimulation can be provided to the diaphragm and/or one or more nerves connected to the diaphragm of the user 10C. The electrical stimulation can aid in stopping the second type of apnea event (e.g., by causing the diaphragm to contract and cause the user 10C to breathe/suck air into the respiration system).

Further, if the control system 330 determines that the user 10C is currently experiencing the first type of apnea event and the second type of apnea event at the same time, the control system 330 (i) causes the first stimulator 304A to provide electrical stimulation to the one or more muscles and/or one or more nerves of the user 10C that are adjacent to the throat 20C of the user 10C and (ii) causes the second stimulator 304B to provide electrical stimulation to the diaphragm and/or one or more nerves connected to the diaphragm of the user 10C.

One or more elements or aspects or steps, or any portion(s) thereof, from one or more of any of claims 1-75 below can be combined with one or more elements or aspects or steps, or any portion(s) thereof, from one or more of any of the other claims 1-75 or combinations thereof, to form one or more additional implementations and/or claims of the present disclosure.

While the present disclosure has been described with reference to one or more particular embodiments and implementations, those skilled in the art will recognize that many changes may be made thereto without departing from the spirit and scope of the present disclosure. Each of these embodiments and implementations and obvious variations thereof is contemplated as falling within the spirit and scope of the present disclosure, which is set forth in the claims that follow.

1-13. (canceled)

14. A system for aiding a user, the system comprising:
a housing configured to be positioned in the user adjacent to an airway of the user;
a stimulator coupled to the housing;
a receiver coupled to the housing;
a collar configured to be worn around a neck of the user;
a transmitter coupled to the collar and being configured to communicate with the receiver to cause the stimulator to selectively provide electrical stimulation to (i) one or more muscles of the user that are adjacent to the airway (ii) one or more nerves associated with the one or more muscles, or (iii) both (i) and (ii);
a sensor configured to generate data associated with the airway of the user;
a memory storing machine-readable instructions; and
a control system including one or more processors configured to execute the machine-readable instructions to:
analyze the generated data to determine (i) if the user is experiencing an apnea event, (ii) if the user is about to experience an apnea event, (iii) if the user is no longer experiencing an apnea event, (iv) or any combination thereof; and
in response to a determination that (i) the user is experiencing an apnea event or (ii) the user is about to experience an apnea event, cause the transmitter to communicate with the receiver such that the stimulator provides the electrical stimulation to aid in stopping or preventing the apnea event.

15-19. (canceled)

20. The system of claim 14, wherein the control system is further configured to execute the machine-readable instructions to analyze the generated data to determine a sleep state of the user, a tension of the one or more muscles, or both.

21. The system of claim 14, wherein the control system is further configured to execute the machine-readable instructions to vary one or more parameters of the electrical stimulation, the one or more parameters of the electrical stimulation including frequency, intensity, duration, dwell time, rise time in a pulse, a ratio of on-time to an off-time, or any combination thereof, wherein the one or more parameters of the electrical stimulation are varied based on a measured response of the one or more muscles to the electrical stimulation.

22-23. (canceled)

24. The system of claim 14, wherein the control system is further configured to execute the machine-readable instructions to (i) automatically increase an intensity of the electrical stimulation when the stimulator provides the electrical stimulation and (ii) analyze the generated data to determine if a current level of the automatically increased intensity of the electrical stimulation has caused the one or more muscles to contract.

25. (canceled)

26. The system of claim 24, wherein the control system is further configured to execute the machine-readable instructions to:
continue automatically increasing the intensity of the electrical stimulation beyond the current level in response to a determination that the current level has not caused the one or more muscles to contract; and
stop automatically increasing the intensity of the electrical stimulation at the current level in response to a determination that the current level has caused the one or more muscles to contract.

27. The system of claim 14, wherein the stimulator includes two or more leads at least partially protruding from the housing, wherein a first one of the two or more leads is configured to provide the electrical stimulation at a first frequency to a first one of the one or more muscles and a second one of the two or more leads is configured to provide the electrical stimulation at a second frequency a second one of the one or more muscles.

28-31. (canceled)

32. The system of claim 14, wherein the sensor is a motion sensor configured to detect motion of the airway.

33. The system of claim 14, wherein the sensor is a photoplethysmography (PPG) sensor and the data is indicative of blood flow of the user adjacent to the airway, blood oxygen levels of the user adjacent to the airway, heart rate of the user, an apnea event the user is currently experiencing, an apnea event the user is likely to experience in the future, or any combination thereof.

34. The system of claim 14, wherein the sensor is a microphone and the data is sound data indicative of snoring, choking, an apnea event, or any combination thereof.

35. The system of claim 14, wherein the sensor includes a motion sensor, a photoplethysmography (PPG) sensor, a blood oxygen sensor, a blood flow sensor, a microphone, a skin conductance sensor, a pulse sensor, a respiration sensor, an EKG sensor, an EMG sensor, an airflow sensor, or any combination thereof.

36-37. (canceled)

38. The system of claim 14, further comprising a battery coupled to the housing and being configured to supply power to the stimulator, wherein the collar is configured to wireless charge the battery.

39-47. (canceled)

48. A system for aiding a user, the system comprising:
a stimulator configured to be positioned in the user adjacent to an airway of the user;
a sensor configured to generate data associated with the airway of the user;
an external device configured to wirelessly power the stimulator;
a memory storing machine-readable instructions; and
a control system including one or more processors configured to execute the machine-readable instructions to:
determine, based at least on an analysis of the generated data, that the user is currently experiencing an apnea event; and
in response to the determination that the user is currently experiencing an apnea event, cause the stimulator to provide electrical stimulation, at a first intensity level, to one or more muscles of the user that are adjacent to the airway to aid in stopping the apnea event.

49. The system of claim 48, wherein the control system is further configured to execute the machine-readable instructions to analyze the generated data to determine if the first intensity level of the electrical stimulation has caused the one or more muscles to contract.

50. The system of claim 49, wherein the control system is further configured to execute the machine-readable instructions to:
automatically increase the intensity of the electrical stimulation beyond the first intensity level in response to a determination that the first intensity level has not caused the one or more muscles to contract; and
stop automatically increasing the intensity of the electrical stimulation at a second intensity level in response to a determination that the second level has caused the one or more muscles to contract.

51. (canceled)

52. The system of claim 48, wherein the stimulator is an electrical conductor and has a length between about 1 millimeter and about 10 millimeters and a diameter between about 0.1 millimeters and about 2 millimeters.

53-55. (canceled)

56. The system of claim 48, wherein the external device includes a magnetic field generator.

57. (canceled)

58. The system of claim 48, wherein the external device includes a collar configured to be worn around a neck of the user.

59. The system of claim 48, wherein the external device includes a stretchable band configured to be worn around a chest of the user, and wherein the sensor includes a strain gauge, an accelerometer, or both.

60. (canceled)

61. The system of claim 48, wherein the external device includes a patch configured to be worn on skin of the user.

62. The system of claim 48, further comprising a housing configured to be positioned in the user adjacent to the airway of the user, wherein the stimulator, the sensor, the memory, and the control system are coupled to the housing such that the sensor, the memory, and the control system are also configured to be positioned in the user.

63-75. (canceled)
